SEQUENCE LISTING (6 sequences)

SEQ ID NO: 1 - DNA, 20 bp, Artificial Sequence
  thiolated bar code DNA strand
ggtaagcatcgaggtaagca

SEQ ID NO: 2 - DNA, 20 bp, Artificial Sequence
  FAM (6-carboxyfluorescine)-labeled bar code DNA strand
tgcttacctcgatgcttacc

SEQ ID NO: 3 - DNA, 30 bp, Artificial Sequence
  thiolated bar code DNA strand
aaaaaaaaaaaaaaataccacatcatccat

SEQ ID NO: 4 - DNA, 30 bp, Artificial Sequence
  FAM (6-carboxyfluorescine)-labeled bar code DNA strand
atggatgatgtggtattttttttttttttt

SEQ ID NO: 5 - DNA, 40 bp, Artificial Sequence
  thiolated bar code DNA strand
aaaaaaaaaaaaaaatacctactacaaaataaaaaaaaaa

SEQ ID NO: 6 - DNA, 40 bp, Artificial Sequence
  FAM (6-carboxyfluorescine)-labeled bar code DNA strand
ttttttttttattttgtagtaggtattttttttttttttt

(19) United States
(12) Patent Application Publication
(10) Pub. No.: US 2013/0130364 A1
(43) Pub. Date: May 23, 2013

(54) MICRODEVICE FOR PATHOGEN DETECTION

(71) Applicants: Tae Seok Seo, Daejeon (KR); Jae Hwan Jung, Daejeon (KR)

(72) Inventors: Tae Seok Seo, Daejeon (KR); Jae Hwan Jung, Daejeon (KR)

(73) Assignee: KOREA ADVANCED INSTITUTE OF SCIENCE AND TECHNOLOGY, Daejeon (KR)

(21) Appl. No.: 13/593,726

(22) Filed: Aug. 24, 2012

(30) Foreign Application Priority Data
Nov. 22, 2011 (KR) 10-2011-0122199

(51) Int. Cl. C12M 1/34; B82Y 15/00

(52) U.S. Cl. 435/287.2; 977/773; 977/810

(57) ABSTRACT

There is provided a microdevice for biomaterial detection, including a passive micromixer to mix a biomaterial, a first probe, and a second probe; a magnetic separation chamber connected with the passive micromixer; and a capillary electrophoresis channel connected with the magnetic separation chamber.

### CROSS-REFERENCE TO RELATED APPLICATION

This application claims the benefits of Korean Patent Application No. 10-2011-0122199 filed Nov. 22, 2011. The entire disclosure of the prior application is incorporated herein by reference in its entirety.

### TECHNICAL FIELD

The present disclosure relates to a microdevice for biomaterial detection including a passive micromixer, a magnetic separation chamber, and a capillary electrophoresis channel.

### BACKGROUND ART

Laboratory-on-a-chip (LOC) technology has continuously progressed by incorporating several chemical and biological functional units into a single wafer. Microfluidics-based miniaturization and integration has brought a number of advantages such as short analysis time, reduced sample consumption, high detection sensitivity, automation and portability. There have been conducted various related researches such as “Lab-on-a-chip having capillary valve and method for manufacturing capillary valve for lab-on-a-chip” (Korean Patent Publication No. 10-2010-0071217).

Current researches related to the LOC technology have moved toward embedding a sample preparation step on a chip to realize a fully integrated LOC for point-of-care (POC) testing. Applicability of LOC to the fields of biological diagnostics and high-throughput bio/chemical screening has already been proved. Pathogen analysis on a chip has especially attracted attention due to the dramatically increasing threat of infectious disease for the public. Therefore, rapid and accurate on-site pathogen diagnostics are demanded, and for this purpose, researches on various molecular assays such as polymerase chain reaction (PCR), microarray, or enzyme-linked immune-sorbent assay (ELISA) have been conducted.

Currently, however, integration of sample pretreatment units and reduction of assay time are still required. Further, detection sensitivity should be improved to a single cell level in consideration of an infectious dose of pathogens such as E. coli o157. Further, simplification of the design of POC pathogen detection system, increase of the speed of a bioassay reaction, improvement of detection sensitivity are also required.

### DISCLOSURE OF THE INVENTION

### Problems to be Solved by the Invention

The present inventors have found out that by using an integrated microdevice for biomaterial detection in accordance with the present disclosure, it is possible to meet such various requirements currently required for the LOC technology, such as more simplified design for the pathogen detection system, high speed bioassay reaction and higher sensitivity.

The present disclosure provides a microdevice for biomaterial detection including a passive micromixer to mix a biomaterial, a first probe, and a second probe; a magnetic separation chamber connected with the passive micromixer; and a capillary electrophoresis channel connected with the magnetic separation chamber.

However, the problems sought to be solved by the present disclosure are not limited to the above description and other problems can be clearly understood by those skilled in the art from the following description.

### Means for Solving the Problems

In accordance with a first aspect of the present disclosure, there is provided a microdevice for biomaterial detection including a passive micromixer to mix a biomaterial, a first probe, and a second probe; a magnetic separation chamber connected with the passive micromixer; and a capillary electrophoresis channel connected with the magnetic separation chamber.

### Effect of the Invention

In accordance with an illustrative embodiment, there is provided a microdevice for biomaterial detection having a simple and integrated structure. The microdevice includes a highly efficient micromixer, a magnetic separation chamber, and a capillary electrophoresis microchannel. By using the microdevice, it is possible to perform on-site detection of a biomaterial from a clinical or environmental sample with a sample-in-answer-out ability. Thus, the microdevice has a wide range of applications to, e.g., biosafety test, environment screening, and clinical trial.

The microdevice in accordance with the illustrative embodiment can be used for, but not limited to, a monoplex biomaterial detection for one kind of biomaterial, and a multiplex biomaterial detection for at least two kinds of biomaterials. By way of non-limiting example, by using the microdevice for biomaterial detection of the illustrative embodiment, a multiplex biomaterial detection for three kinds of target pathogens (Staphylococcus aureus, Escherichia coli O157:H7, and Salmonella typhimurium) can be successively performed. The fully integrated microdevice in accordance with the illustrative embodiment has a sample-in-answer-out ability and is capable of detecting a multiplex biomaterial with high sensitivity. Accordingly, the microdevice can be applied to, but not limited to, point-of-care (POC) testing for diagnosing a disease.

Using the microdevice for biomaterial detection in accordance with the illustrative embodiment has an advantage in that more rapid analysis can be conducted as compared to conventional analysis methods. By way of example, it takes about 20 minutes to form immune-complex by using the passive micromixer of the microdevice, less than about 5 minutes to implement magnetic separation and dehybridization of barcode DNAs in the magnetic separation chamber of the microdevice, and less than about 5 minutes to separate and detect barcode DNA strands in the capillary electrophoresis channel of the microdevice by using an electrophoresis method. Accordingly, a total analysis time may be less than about 30 minutes, much shorter than analysis times for conventional analysis methods.

Further, by using the microdevice for biomaterial detection in accordance with the illustrative embodiment, it is still possible to detect a biomaterial even when the concentration of the biomaterial is less than about 10⁵ CFU (Colony Forming Unit). For example, in order to detect a pathogen such as E. coli O157, detection sensitivity needs to be improved to a single-cell level in consideration of an infectious dose of the pathogen. The microdevice in accordance with the illustrative embodiment can perform the detection efficiently while satisfying such requirement for the detection sensitivity. Further, the microdevice in accordance with the illustrative embodiment also has an advantage in that the detection can be performed at a single-cell level.

### BRIEF DESCRIPTION OF THE DRAWINGS

Non-limiting and non-exhaustive embodiments will be described in conjunction with the accompanying drawings. Understanding that these drawings depict only several embodiments in accordance with the disclosure and are, therefore, not to be intended to limit its scope, the disclosure will be described with specificity and detail through use of the accompanying drawings, in which:

FIG. 1 is a schematic diagram illustrating a microdevice for biomaterial detection including a passive micromixer, a magnetic separation chamber, and a capillary electrophoresis channel manufactured in accordance with an illustrative embodiment;

FIG. 2 provides experiment results for investigating optimum amounts of antibodies to be conjugated with AuNP (Gold Nano Particle) probes in accordance with an illustrative embodiment: FIGS. 2a to 2c relate to a monoclonal anti-Staphylococcus aureus, a monoclonal anti-E. coli O157:H7, and a monoclonal anti-Salmonella typhimurium, respectively;

FIG. 3 is a schematic diagram for illustrating effective mixing that occurs at a passive micromixer of a microdevice for biomaterial detection manufactured in accordance with an illustrative embodiment;

FIG. 4 is a graph showing retention time obtained as a result of an experiment for relative cell capture efficiency with about 10⁵ of CFU Staphylococcus aureus in accordance with an illustrative embodiment;

FIG. 5 provides electrophoregrams showing monoplex pathogen detection results in accordance with an illustrative embodiment: FIGS. 5a to 5c relate to Staphylococcus aureus, E. coli O157:H7, and Salmonella typhimurium, respectively;

FIG. 6 is a graph showing measurements of RFU (Relative Fluorescent Unit) as a function of a target pathogen concentration (pathogen CFU) in an experiment of monoplex pathogen detection in accordance with an illustrative embodiment;

FIG. 7 is a graph showing measurements of RFU in multiplex pathogen detection for detecting multiplex pathogens including (i) Staphylococcus aureus+E. coli O157:H7, (ii) Staphylococcus aureus+Salmonella typhimurium, (iii) E. coli O157:H7+Salmonella typhimurium, and (iv) Staphylococcus aureus+E. coli O157:H7+salmonella typhimurium, wherein the concentration of each pathogen is about 10⁵ CFU; and

FIG. 8 is a graph showing a result of a LOD (Limit of Detection) test using a microdevice in accordance with an illustrative embodiment, wherein peaks on the graph from the left indicate the presence of Staphylococcus aureus, E. coli O157:H7, and Salmonella typhimurium in order.

### BEST MODE FOR CARRYING OUT THE INVENTION

Hereinafter, illustrative embodiments and examples will be described in detail so that inventive concept may be readily implemented by those skilled in the art.

However, it is to be noted that the present disclosure is not limited to the illustrative embodiments and examples but can be realized in various other ways. In drawings, parts irrelevant to the description are omitted for the simplicity of explanation, and like reference numerals denote like parts through the whole document.

Through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “on” that is used to designate a position of one element with respect to another element includes both a case that the one element is adjacent to the another element and a case that any other element exists between these two elements.

Through the whole document, the term “combinations of” included in Markush type description means mixture or combination of one or more components, steps, operations and/or elements selected from the group consisting of components, steps, operation and/or elements described in Markush type and thereby means that the disclosure includes one or more components, steps, operations and/or elements selected from the Markush group.

The term “about or approximately” or “substantially” are intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Hereinafter, illustrative embodiments and examples will be explained in detail with reference to the accompanying drawings.

In accordance with a first aspect of the present disclosure, there is provided a microdevice for biomaterial detection including a passive micromixer to mix a biomaterial, a first probe, and a second probe; a magnetic separation chamber connected with the passive micromixer; and a capillary electrophoresis channel connected with the magnetic separation chamber.

In accordance with an illustrative embodiment, in the microdevice for biomaterial detection of the first aspect, a biomaterial can be detected by using its specific antibody, but the illustrative embodiment is not limited thereto. By way of non-limiting example, the microdevice for biomaterial detection in accordance with the first aspect of the present disclosure may be used to detect various kinds of biomaterials that have specific antibodies such as a bacterial pathogen, a viral pathogen, various kinds of cells and various kinds of proteins and thus can be detected by using a specific reaction between an antigen and an antibody. In the following description, the microdevice for biomaterial detection will be described for the example case of detecting a bacterial pathogen. However, it should be noted that the present disclosure is not limited thereto.

By way of example, the bacterial pathogen may include, but not limited to, at least one pathogen selected from the group consisting of Staphylococcus aureus, Eschericia coli (E. coli) O157:H7, and Salmonella typhimurium. For example, in the event that the pathogen is of one kind, a monoplex pathogen detection may be performed, and in the event that the pathogen is of more than one kind, a multiplex pathogen detection may be performed. For example, the pathogen may be, but not limited to, all kinds of bacteria having antibodies. Further, the microdevice for biomaterial detection in accordance with the present disclosure may also be applicable to the detection of, but not limited to, all kind of cancer cells and other proteins as well as the detection of the all kinds of bacteria having antibodies.

In accordance with an illustrative embodiment, the first probe may include a magnetic microparticle (MMP) probe, but not limited thereto. By way of example, if the first probe is a MMP probe, it may become easier to separate the first probe by using the magnetic separation chamber of the microdevice of the present disclosure.

In accordance with an illustrative embodiment, the MMP probe may include, but not limited to, at least one specific antibody for the biomaterial. Here, the specific antibody may be immobilized at the surface of the MMP probe, but not limited thereto. For example, the specific antibody immobilized at the surface of the MMP probe may be of one kind and plural in number.

Regarding the antibody, experimentally, it is known that, for the improvement of efficiency, it will be helpful to immobilize a monoclonal antibody to a magnetic particle such as MMP, whereas it will be helpful to immobilize a polyclonal antibody to a metallic nanoparticle such as AuNP (Gold Nanoparticle). Accordingly, the MMP probe may include, at the surface thereof, an immobilized specific monoclonal antibody for the biomaterial. However, it should be noted that the present disclosure is not limited to this example.

In accordance with an illustrative embodiment, the second probe may include nanoparticles of, but not limited to, gold, silver, platinum, palladium, copper, nickel, zinc, or silicon oxide. By way of example, the second probe may include an AuNP (Gold Nanoparticle) probe, but not limited thereto. Besides the AuNP, the second probe may include all kinds of nanoparticles to which polymer can be coupled.

In accordance with an illustrative embodiment, the nanoparticle may include, but not limited to, a specific antibody for the biomaterial and at least one barcode polymer. Each of the specific antibody and the barcode polymer may be immobilized at a surface of the nanoparticle, but not limited thereto.

In accordance with an illustrative embodiment, the barcode polymer may have a negative charge, and may be available to be separated according to its size by using a capillary electrophoresis, but not limited thereto. By way of example, the barcode polymer may include a barcode DNA having the negative charge, but not limited thereto.

By way of non-limiting example, the barcode DNA may include a FAM (6-carboxy-fluorescine) label at the 5′ end. Further, for example, the size of the barcode DNA strand may differ depending on the kind of a target bacterial pathogen. Accordingly, during electrophoresis, an elution time of peaks of barcode DNAs appearing on an electrophoregram may differ depending on the kind of the target bacterial pathogen. By using this, the microdevice in accordance with the present disclosure can be applied to, but not limited to, not only the monoplex pathogen detection but also the multiplex pathogen detection. By way of non-limiting example, the specific antibody for the biomaterial immobilized at the surface of the nanoparticle may be of one kind and plural in number. Further, the barcode DNA immobilized at the surface of the nanoparticle may also be of one kind and plural in number. However, it should be noted that the present disclosure is not still limited thereto.

Regarding the antibody, experimentally, it is known that, for the improvement of efficiency, it will be helpful to immobilize a monoclonal antibody to a magnetic particle such as MMP, whereas it will be helpful to immobilize a polyclonal antibody to a metallic nanoparticle such as AuNP (Gold Nanoparticle). Accordingly, the nanoparticle may include, at the surface thereof, an immobilized specific polyclonal antibody for the biomaterial. However, it should be noted that the present disclosure is not limited to this example.

In accordance with an illustrative embodiment, the passive micromixer may have an intestine-shaped structure including at least one corner and a tooth-shaped projection, and a centrifugal force generated at the corner can improve a mixing efficiency of the passive micromixer. However, the illustrative embodiment is not limited thereto.

In accordance with an illustrative embodiment, the passive micromixer may mix the biomaterial, the first probe, and the second probe to thereby form a complex of first probe-biomaterial-second probe, but the illustrative embodiment is not limited thereto. By way of example, the complex of first probe-biomaterial-second probe may be moved into the magnetic separation chamber, whereas the first probe, the biomaterial and the second probe failing to form the complex may be removed through a cleaning process or the like. However, the illustrative embodiment is not still limited thereto.

In accordance with an illustrative embodiment, the magnetic separation chamber may separate a part of the complex of first probe-biomaterial-second probe formed by applying a magnetic field, but the illustrative embodiment is not limited thereto. By way of example, a separated part of the complex may be a dehybridized strand of a barcode DNA immobilized at the surface of a nanoparticle of the second probe, but not limited thereto. For example, while the magnetic separation chamber is being heated by a heater, a barcode DNA included in the complex of first probe-biomaterial-second probe may be dehybridized. Then, if a magnetic field is applied later, the complex except the dehybridized barcode DNA strand may be captured by the magnetic field. Afterward, if a high-voltage power is supplied, only the hyhybridized barcode DNA strand may be separated and moved toward the capillary electrophoresis channel. However, the illustrative embodiment is not limited thereto.

In accordance with an illustrative embodiment, the capillary electrophoresis channel may quantitatively detect the part of the complex of first probe-biomaterial-second probe separated in the magnetic separation chamber by using a capillary electrophoresis, but not limited thereto. By way of example, the part of the complex separated in the magnetic separation chamber may be, but not limited to, the dehybridized barcode DNA strand. In case that the separated part of the complex is the dehybridized barcode DNA strand, various methods may be employed to analyze it. Among the methods, a capillary electrophoresis (CE) method implemented on a microchip is superior to a DNA hybridization method in that this method enables precise, simple, and rapid quantitative analysis. Since elution times of peaks that appear on the electrophoregram may be affected by DNA sizes, target DNAs can be easily recognized. Further, elution with single base resolution on a chip enables analysis of multiple DNA molecules. For these advantages, the genetic analysis based on the micro capillary electrophoresis may have wide range of applications such as STR (Short Tandem Repeat) genotyping, DNA sequencing and SNP (Single Nucleotide Polymorphism) analysis. In accordance with the present disclosure, it is possible to perform a quantitative detection of barcode DNA strands that are eluted by using the capillary electrophoresis channel. However, the present illustrative embodiment is not limited thereto.

By way of example, the capillary electrophoresis channel included in the microdevice of the present disclosure may have a cross-injector design, but not limited thereto. By way of non-limiting example, the capillary electrophoresis channel having the cross-injector design may have a width of, e.g., about 140 μm and a depth of, e.g., about 40 μm. Moreover, the capillary electrophoresis channel may have an anode and a cathode at both ends thereof, but not limited thereto.

In accordance with an illustrative embodiment, the microdevice for biomaterial detection may further include a sample inlet at a upstream of the passive micromixer, and the biomaterial, the first probe, and the second probe may be introduced into the microdevice through the sample inlet. However, the illustrative embodiment is not limited thereto.

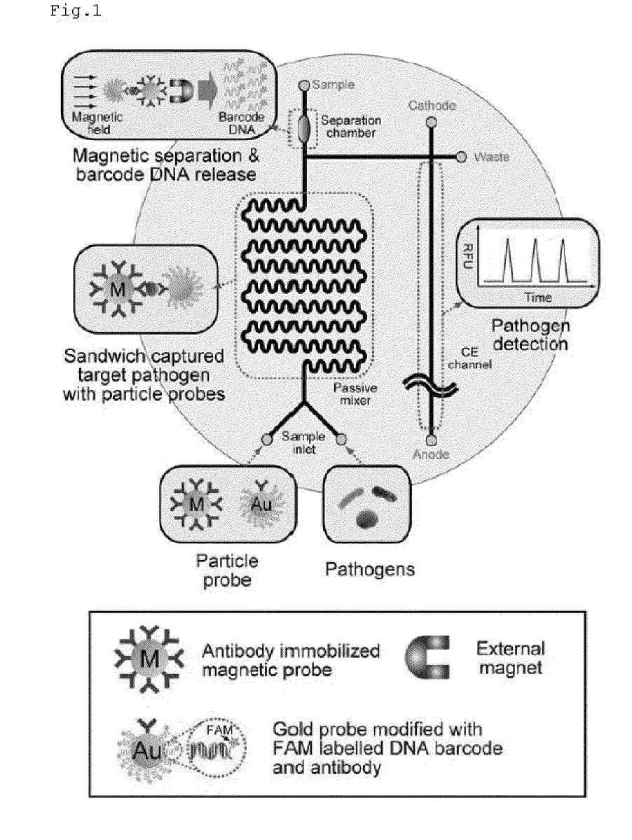

In accordance with an illustrative embodiment, the microdevice for biomaterial detection may further include a sample reservoir and a waste reservoir which are respectively connected with the magnetic separation chamber, and a cathode reservoir and an anode reservoir which are respectively connected with the capillary electrophoresis channel, but not limited thereto. By way of example, each of the sample reservoir and the waste reservoir may be directly connected with one end of the magnetic separation chamber or may be indirectly connected with one end of the magnetic separation chamber via a conduit or the like, as depicted in FIG. 1, but not limited thereto. The waste reservoir may store materials other than a material introduced into the capillary electrophoresis channel in the microdevice. However, the illustrative embodiment is not limited thereto. Meanwhile, by way of example, each of the cathode reservoir and the anode reservoir may be directly connected with one end of the magnetic separation chamber or may be indirectly connected with one end of the magnetic separation chamber via a conduit or the like, but not limited thereto. The electrophoresis microdevice including the sample reservoir, the waste reservoir, the cathode reservoir, and the anode reservoir may be referred to as an “electrophoresis microdevice of a cross-injector design”. However, the illustrative embodiment is not limited thereto.

In accordance with an illustrative embodiment, the microdevice can be used for, but not limited to, a monoplex biomaterial detection for one kind of biomaterial by using a single-sized barcode polymer, or a multiplex biomaterial detection for at least two kinds of biomaterials by using differently-sized barcode polymers. By way of non-limiting example, by using the microdevice for biomaterial detection of the illustrative embodiment, a multiplex biomaterial detection for three kinds of target pathogens (Staphylococcus aureus, Escherichia coli O157:H7, and Salmonella typhimurium) can be successively performed. The fully integrated microdevice in accordance with the illustrative embodiment has a sample-in-answer-out ability and is capable of detecting a multiplex biomaterial with high sensitivity. Accordingly, the microdevice can be applied to, but not limited to, point-of-care (POC) testing for diagnosing a disease.

In accordance with an illustrative embodiment, a total analysis time from sample pretreatment to biomaterial detection by using the microwave device may be, e.g., about 30 minutes or less, but not limited thereto. By way of example, it may take about 20 minutes to form immune-complex by using the passive micromixer of the microdevice, less than about 5 minutes to implement magnetic separation and dehybridization of barcode DNAs in the magnetic separation chamber of the microdevice, and less than about 5 minutes to separate and detect barcode DNA strands in the capillary electrophoresis channel of the microdevice by using the electrophoresis method. Accordingly, a total analysis time may be less than about 30 minutes. However, the illustrative embodiment is not limited thereto. By way of example, a total analysis time for detecting biomaterial by using the microdevice may be, but not limited to, less than about 20 minutes, less than about 25 minutes, or less than about 30 minutes. Using the microdevice for biomaterial detection in accordance with the illustrative embodiment has an advantage in that more rapid analysis can be conducted as compared to conventional analysis methods.

In accordance with an illustrative embodiment, the microdevice may perform the detection at a single-cell level, but not limited thereto. By way of non-limiting example, the microdevice can detect a biomaterial when a concentration of the biomaterial is equal to or less than about 10⁵ CFU, equal to or less than about 10⁴ CFU, equal to or less than about 10³ CFU, equal to or less than about 10² CFU, equal to or less than about 10 CFU, or equal to or less than about 1 CFU, but not limited thereto. When the concentration of the biomaterial is about 1 CFU, the biomaterial is of a single-cell level. That is, by using the microdevice in accordance with the illustrative embodiment, the detection of the biomaterial can be performed at a single-cell level. For example, in order to detect a pathogen such as E. coli O157, detection sensitivity needs to be improved to a single-cell level in consideration of an infectious dose of the pathogen. The microdevice in accordance with the illustrative embodiment can perform the detection efficiently while satisfying such requirement for the detection sensitivity.

FIG. 1 is a schematic diagram illustrating the microdevice including the passive micromixer, the magnetic separation chamber and the capillary electrophoresis channel in accordance with the present disclosure. As can be seen from FIG. 1, the microdevice has a simple and integrated structure while having improved performance such as rapid bioassay reaction and high sensitivity. By using the microwave for biomaterial detection in accordance with the present disclosure, it is possible to perform an on-site detection of a biomaterial from a clinical or environmental sample with a sample-in-answer-out ability. Thus, the microdevice can be applied to, but not limited to, biosafety test, environment screening, and clinical trial.

By way of non-limiting example, the microdevice for biomaterial detection in accordance with the present disclosure can be applied for the improvement of LOC technology, but not limited thereto. Further, by way of example, the microdevice for biomaterial detection in accordance with the present disclosure may be used for a POC (Point-of-Care) service, but not limited thereto.

Hereinafter, examples will be explained in detail, but the illustrative embodiments are not limited thereto.

### EXAMPLES

### 1. Preparation of Antigens, Antibodies, and Barcode DNAs

In this example, three target bacterial cells (i.e., Staphylococcus aureus (KCTC 1621), E. coli o157:H7 (KCTC 1039), Salmonella typhimurium (KCTC 2054)) were purchased from Korean Collection for Type Cultures (KCTC). These bacterial cells were grown aerobically in a nutrient agar (about 3 g of beef extract, about 5 g of peptone, about 15 g of agar and about 1 L of distilled water) at a temperature of about 37° C.

Further, mouse monoclonal and polyclonal antibodies of Staphylococcus aureus and E. coli were purchased from Millipore (Temecula, Calif., USA) and those of Salmonella typhimurium were purchased from Santa Cruz Biotechnology (Santa Cruz, Calif., USA). It is known in the art that immobilizing a monoclonal antibody at a magnetic particle such as a magnetic microparticle (MMP) contributes to the improvement of detection efficiency, whereas immobilizing a polyclonal antibody at a metallic nanoparticle such as AuNP (Gold NanoParticle) leads to the improvement of detection efficiency. Thus, in this example, both the monoclonal antibody and the polyclonal antibody were used.

Meanwhile, three pairs of thiolated and FAM (6-carboxy-fluorescine)-labeled barcode DNA strands having a double helix structure were used to synthesize AuNP probes. The base sequences of the three pairs of barcode DNAs are as follows:

(1) Staphylococcus aureus (20-mer)
5′-SH-C₆-GGTAAGCATCGAGGTAAGCA-3′
and
5′-FAM-TGCTTACCTCGATGCTTACC-3′
(2) E.coli o157:H7 (30-mer)
5′-SH-C₆-AAAAAAAAAAAAAAATACCACATCATCCAT-3′
and
5′-FAM-ATGGATGATGTGGTATTTTTTTTTTTTTTT-3′
(3) Salmonella typhimurium (40-mer)
5′-SH-C₆-
AAAAAAAAAAAAAAATACCTACTACAAAATAAAAAAAAAA-3′
and
5′-FAM-
TTTTTTTTTTATTTTGTAGTAGGTATTTTTTTTTTTTTTT-3′

### 2. Preparation of First Probes and Second Probes

Particle probes were synthesized according to the previously known protocols. Specific process therefor is as follows.

First, tosyl-activated magnetic beads (Dynabeads M280 Tosyl-activated, dia.=2.8 μm, Invitrogen, Carlsbad, Calif., USA) were covalently linked to the primary amino groups of antibodies.

Then, a MMP (Magnetic MicroParticles) probe was manufactured. For the manufacture of the MMP probe, about 100 μL of MMPs (˜2×10⁸) were washed three times with about 1 mL of borated buffer (about 0.1 M, pH of about 9.5), and, at this time, magnetic separation was performed concurrently. Afterward, the MMPs were re-suspended in about 200 μL of borate buffer containing about 60 μg of antibodies (Ab) (about 3 μg of antibody per 10⁷ MMPs). The conjugation of the MMPs with Ab was carried out at about 37° C. for about 24 hours under vortex. Then, the Ab-conjugated MMPs were placed on a magnet and washed with PBS (about 0.01 M, pH 7.4) for about 5 minutes at about 4° C. Subsequently, the MMP probes were passivated by adding about 250 μL of blocking buffer (about 0.2 M Tris, pH 8.5) for about 4 hours at about 37° C. and washed for about 5 minutes at about 4° C. The MMP probes were stored in about 1 mL of PBS at about 4° C. before they are used.

The coupling efficiency between the MMPs and the antibodies was measured based on an absorbance at about 280 nm before and after the reaction [coupling efficiency (%)={(A_{280,before} A_{280,after}) (A_{280,before})}×100]. For about 2×10⁸ of MMPs, the loaded amount of each Ab was about 50.9 μg for monoclonal anti-Staphylococcus aureus, about 56.4 μg for monoclonal anti-E. coli O157:H7, and about 34.4 μg for monoclonal anti-Salmonella typhimurium with the coupling efficiency of about 84.8%, about 93.9%, and about 57.1%, respectively.

Then, the AuNP (Gold Nanoparticle) probes were prepared by adding Ab to about 0.1 mL of AuNP solution (about 2×10¹¹ mL⁻¹=about 330 fmoles mL⁻¹, dia.=about 30 nm, BBInternational, UK) at pH 9.2.

The amount of each Ab for the conjugation with AuNPs was roughly estimated as about 100 ng for Staphylococcus aureus, about 300 ng for E. coli O157:H7, and about 100 ng for Salmonella typhimurium when the amount of the AuNPs was set to about 2×10¹⁰.

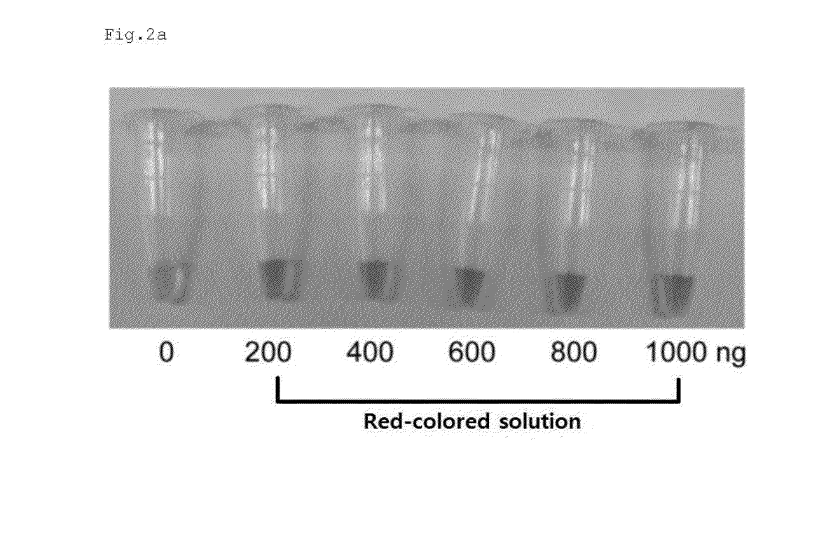

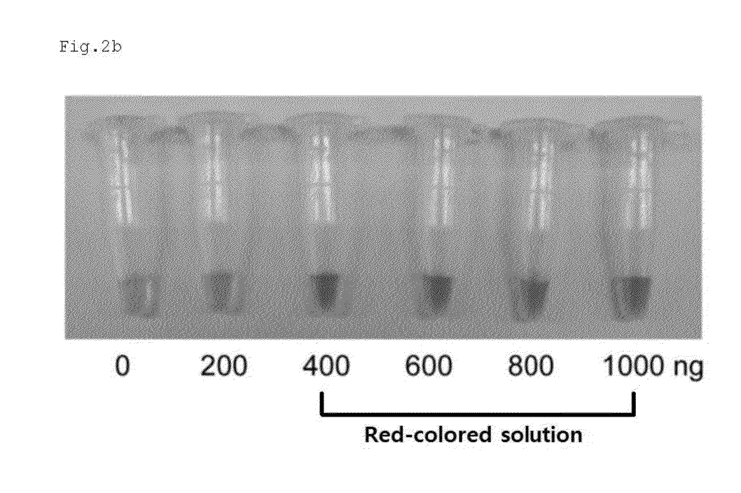

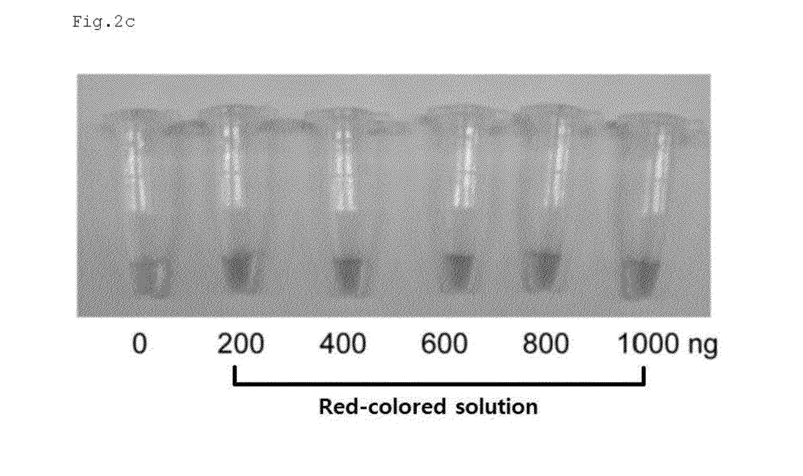

FIG. 2 provides an experimental result of investigating an optimum amount of antibodies to be conjugated with the AuNP probes. To elaborate, FIG. 2a provides a result of monoclonal anti-Staphylococcus aureus; FIG. 2b, monoclonal anti-E. coli O157:H7; and FIG. 2c, monoclonal anti-Salmonella typhimurium. To be more specific, in FIG. 2, red-shifts represent AuNP condensation induced by NaCl (about 2M, about 10 μL), and they are substantially used as labels that indicate how much area the AuNPs are capable of providing in order to be coupled with thiolated barcode DNAs. By way of example, AuNPs without having antibodies were condensed, showing red-shifts, whereas AuNPs conjugated with antibodies were stable and were not condensed. That is, if a sufficient amount of antibodies are immobilized at the surfaces of AuNPs, condensation of the particles is prevented, which also implies that surface areas to be coupled with thiolated barcode DNAs are not sufficient. From the results of FIGS. 2a to 2c, it was proved that for about 0.1 mL of AuNP, an optimum amount of antibodies to be conjugated with the AuNP probes is about 100 ng for monoclonal anti-Staphylococcus aureus, about 300 ng for monoclonal anti-E. coli O157:H7, and about 100 ng for monoclonal anti-Salmonella typhimurium.

In order to modify the AuNPs with an optimum amount of antibodies, the AuNPs were incubated at a room temperature for about 30 minutes under slow vortex by using a Dynabeads Sample Mixer. Then, the Ab modified AuNPs were reacted with the newly cleaved thiolated barcode DNA strands (1 nmole) for about 16 hours. The thiolated barcode DNAs were prepared by reducing the protecting disulfide bond to thiol group through treatment with dithiothreitol (DTT, Sigma-Aldrich, Mo., USA) and purified through illustra NAP-5 columns (GE Healthcare, NJ, USA). Next, the AuNPs were salt-stabilized with about 0.1 M of NaCl and passivated with about 1% of BSA solution for about 30 minutes. Then, the AuNPs were centrifuged at about 13 000 rpm for about 1 hour at about 4° C. and the supernatant was removed. This washing step was repeated twice. Subsequently, the AuNPs were re-suspended in PBS and then hybridized with the FAM-labeled complementary barcode DNA strands for about 6 hours at about 37° C. The Ab and the duplex barcode DNA labeled AuNPs were purified again through a centrifugation procedure and re-dispersed in about 200 mL of washing buffer (i.e., PBS containing about 0.1% of BSA and about 0.02% of Tween 20). The prepared AuNP probes were stored at a low temperature of about 4° C. prior to use.

As for the AuNP probes, the loading amount of DNA was determined based on the absorbance at about 260 nm. The numbers of barcode DNA complements per about 2×10¹⁰ of AuNP AuNP probes were about 0.368, about 0.377, and about 0.434 nmoles, which correspond to about 1.11×10⁴, about 1.13×10⁴, and about 1.31×10⁴ of barcode DNA strands per each AuNP, respectively.

### 3. Fabrication of Microdevice for Biomaterial Detection

The microdevice for biomaterial detection in accordance with the present disclosure included, as depicted in FIG. 1, three parts: a passive mixer, a magnetic separation chamber, and a capillary electrophoresis (CE) microchannel.

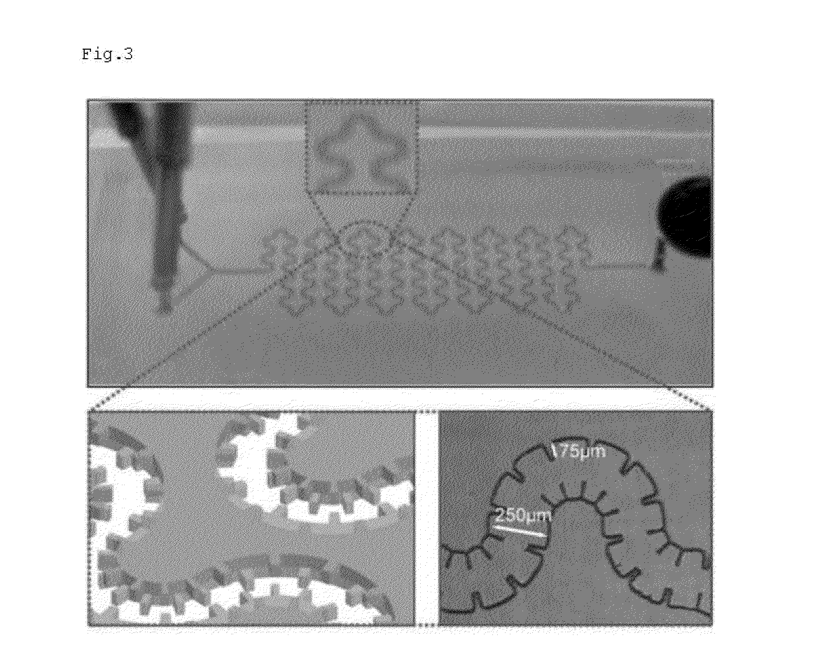

As shown in FIG. 3, the passive mixer had an intestine-shaped serpentine 3D structure to allow an effective mixing of a pathogen, a first probe, and a second probe and trigger an immuno-binding reaction therebetween to thereby form a complex of pathogen-first probe-second probe. In this example, the passive micromixer had a length about 17.9 cm, a width of about 250 μm, and a height of about 100 μm. A total volume of the passive micromixer was about 3.80 μL.

The magnetic separation chamber had a volume of about 1.8 mL and was sandwiched between an external magnet on top of it and a film heater underneath it. Only a barcode DNA plug was separated and generated from the complex of pathogen-first probe-second probe in the separation chamber. The barcode DNA plug traveled down toward the CE microchannel having a cross-injector design and a separation length of about 6 cm.

The passive micromixer integrated microdevice was made of a glass-glass wafer. To form a passive micro mixer-magnetic separation chamber-CE microchannel pattern on an upper wafer for forming the glass-glass wafer, about 100 mm of borofloat wafer (having a thickness of about 1.1 mm, PG&O, Santa Ana, Calif., USA) was coated with about 200 nm of amorphous silicon using low-pressure chemical vapor deposition. Thereafter, a photoresist (S1818, Rohm & Haas, Philadelphia, Pa., USA) was spin-coated in a thickness of about 2 μm, and the passive micromixer-magnetic separation chamber-CE microchannel pattern of the mask was transferred through UV exposure. After a developing process, the exposed Si hard mask was removed by reactive ion etching (RIE) in SF₆ plasma (VSRIE-400A, Vacuum Science, Korea). Isotropic wet etching was subsequently performed in about 49% of hydrofluoric acid solution for about 8 minutes to achieve a wafer depth of about 50 μm and a wafer width of about 140 μm. The remaining photoresist was cleaned in acetone for 10 min, and the sacrificial silicon layer was then removed by RIE in SF₆ plasma. Reservoir holes were drilled in a diameter of about 1 mm using a Sherline vertical milling machine (Model 2010, Sherline Products, Vista, Calif., USA)

A passive micromixer-magnetic separation chamber-CE microchannel pattern on a lower wafer was also fabricated by performing the above-described process in a thickness of about 50 μm. Then, the upper and lower wafers were aligned and thermally bonded to each other at a temperature of about 668° C. for about 2 hours, to thereby obtain the glass-glass wafer. Further, a punctuated PDMS membrane (having a diameter of about 3 mm and a thickness of about 3 mm) was treated in a UV-ozone cleaner for about 5 minutes. Then, the sample reservoir, the waste reservoir, the cathode and the anode are assembled for electrode connection, so that the microdevice for biomaterial detection was obtained.

### 4. Passive Micromixer Incorporated in Microdevice for Biomaterial Detection

To maximize the cell capture efficiency of the microdevice for biomaterial detection in accordance with the present disclosure, it is critical to optimize a micromixer and a flow rate. The intestine-shaped serpentine 3D micromixer in accordance with the present disclosure is advantageous due to its high mixing efficiency with high speed derived from a centrifugal force at corners. In addition to the serpentine design, in the micromixer of the present disclosure, a regular tooth-shaped projection was incorporated in the serpentine microchannel to further enhance the mixing efficiency. Each of the upper and lower glass wafers had such a tooth-shaped projection, as shown in the bottom of FIG. 3.

With this structure of the micromixer, a pathogen sample and particle probe solutions can be moved horizontally and vertically, thus allowing formation of immuno-complexes with improved mixing efficiency. The mixing efficiency of the novel passive micromixer was proved by a mixing test using red and blue dyes.

As a result of the mixing test, full mixing of the passive micromixer was achieved after passing 4 mixing units (approximately within a length of about 3.25 cm, which is equivalent to about 25% of the total length) even at a high flow rate of about 5000 μL/h. this result was obtained by observing uniform violet color in the magnified digital image. This test result implies that a lower flow rate could produce better mixing performance. In this regard, cell capture efficiencies at different flow rates were evaluated while controlling the retention time of particle probes and target cells in the passive micromixer.

To elaborate, a cell sample (about 10⁵ CFU of Staphylococcus aureus) was injected with the particle probes and mixed along the microfluidic channel at flow rates ranging from about 3.8 μL/h to about 100 μL/h. The immuno-complexes were then isolated by using a magnet placed on the top of the separation chamber, and, then, barcode DNAs were released by heating the chamber through the use of a rubber heater. Fluorescence signals of the recollected barcode DNAs were quantitatively analyzed by using capillary electrophoresis, and a relative cell capture efficiency was calculated as a relative value for a fluorescence signal (100%) at about 60 minutes of retention time corresponding to a flow rate of 3.8 μL/h.

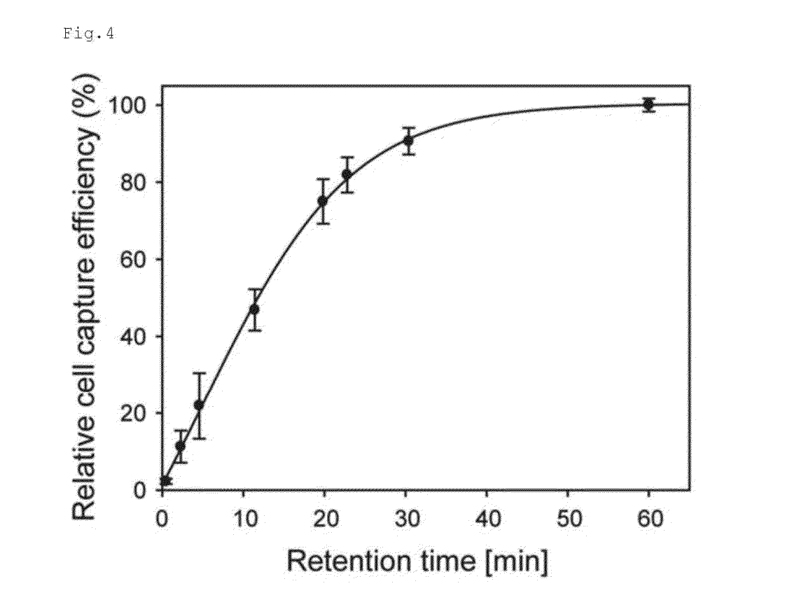

FIG. 4 is a graph showing a retention time as an experimental result of relative cell capture efficiency using about 10⁵ CFU of Staphylococcus aureus. As can be seen from FIG. 4, the cell capture efficiency increases in proportion to the retention time. In particular, about 75% of cells were captured at a retention time of about 20 minutes (i.e., at a flow rate of about 11.5 μL/h).

In view of this experimental result, the retention time was fixed to about 20 minutes for further experiments in order to conduct the whole process of the experiment rapidly as well as to maintain high detection sensitivity for biomaterial.

### 5. Operation of Microdevice for Biomaterial Detection

After the mixing using the passive micromixer, the process of detecting a pathogen by the microdevice is divided into two steps: target pathogen capture using the magnetic separation chamber and barcode DNA detection using the CE microchannel. Those two steps are illustrated in FIG. 1.

First, the CE microchannel was cleaned with about 1M of NaOH for about 10 minutes and with about 1M of HCl for about 3 minutes. Then, the CE microchannel was rinsed with water. Then, the channel was pretreated with v/v dynamic coating (DEH-100, The Gel Company, San Francisco, Calif., USA) mixed with about 50% of methanol for about 2 minutes to minimize electroosmotic flow during separation.

The separation channel was filled with about 5% of linear polyacrylamide (LPA) and about 6 M of urea from the anode reservoir as a sieving matrix. The waste, cathode and anode reservoirs were filled with 1×TTE (Tris TAPS EDTA) buffer.

Next, an aqueous solution containing MMP and AuNP probes (about 10 mL for each) and a sample solution containing target pathogens (about 10 mL) were introduced into the microdevice from the sample inlet by using a syringe pump. The solutions were well mixed by the passive micromixer while they are flown, to thereby form immuno-complexes of a sandwich structure including MMP probe-pathogenic bacteria-DNA barcode labeled AuNP probe. The immuno-complexes were collected on the magnetic separation chamber of the microdevice with a magnet, whereas particle probes and targets that are not bonded together were washed away with PBS (about 0.01 M, pH 7.4).

The FAM-labeled barcode DNA strands were dehybridized from the AuNP probes by heating the magnetic separation chamber with a silicon rubber heater (SR020312, Hanil Electric Heat Engineering, Korea) at a temperature of about 95° C. for about 3 minutes. Then, a high-voltage power was supplied to selectively move the FAM-labeled barcode DNA to the CE microchannel. Afterward, CE operation and laser-induced fluorescence detection were performed according to previously known methods. Briefly, the separation channel was heated with a silicon rubber heater (SR020312, Hanil Electric Heat Engineering, Korea) and maintained at a temperature of about 70° C. while being monitored by a temperature controller (TZ4ST-14S, Autonics, Korea). Power of about 1000 V and about 0 V (PS300 series, Stanford Research Systems) were supplied to the waste and sample reservoirs for about 60 seconds, thereby allowing the released barcode DNA strands to be loaded into the injection channel. To separate a DNA plug at the injection cross, a voltage of about 900 V was applied to the sample and waste reservoirs for about 10 seconds with an electric field strength of about 300 V cm⁻¹ along the separation channel. Then, the CE separation was implemented by applying a voltage of about 1800 V to the anode, during which the sample and waste reservoirs were maintained in floating state. These series of CE operations were controlled automatically by a LabVIEW program.

Fluorescence emission signals of the separated FAM-labeled barcode DNA strands were detected by using a laser-induced confocal fluorescence microscope (Clsi, Nikon, Japan). An excitation wavelength of about 488 nm from an argon laser was used, and the power intensity measured from a 10×Plan Apo objective (NA 0.45) was about 3.6 mW. The scanning area (0.016 mm²) was defined on the separation channel on the side of the anode, and data were obtained with a scanning rate of 5 frames per second. The emission signal of the FAM was detected through a band pass filter of about 505 nm to about 530 nm. Peaks on the electropherogram were quantified using the PeakFit (Version 4.12) software.

### 6. Monoplex Detection

To realize a microdevice for quantitative and sensitive detection of pathogens, it is critical that this novel device should provide good signal response over several orders of magnitude with a low LOD (Limit of Detection) value. Thus, the present inventors have demonstrated the capability of the microdevice to identify the three types of target pathogens, Staphylococcus aureus, E. coli O157:H7, and Salmonella typhimurium, in the range of about 1 CFU to about 10⁶ CFU.

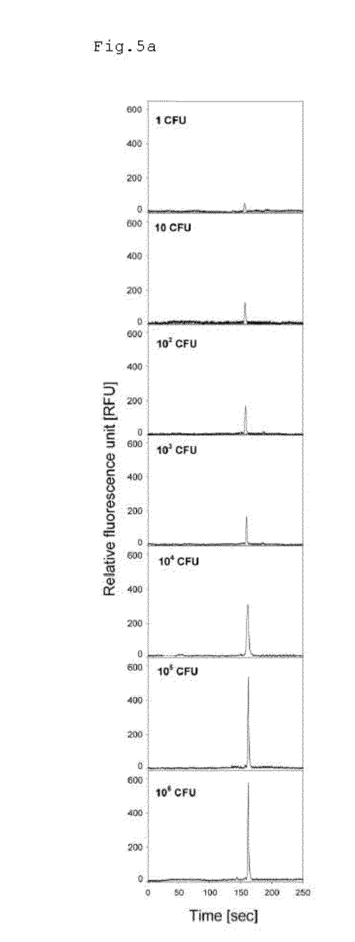

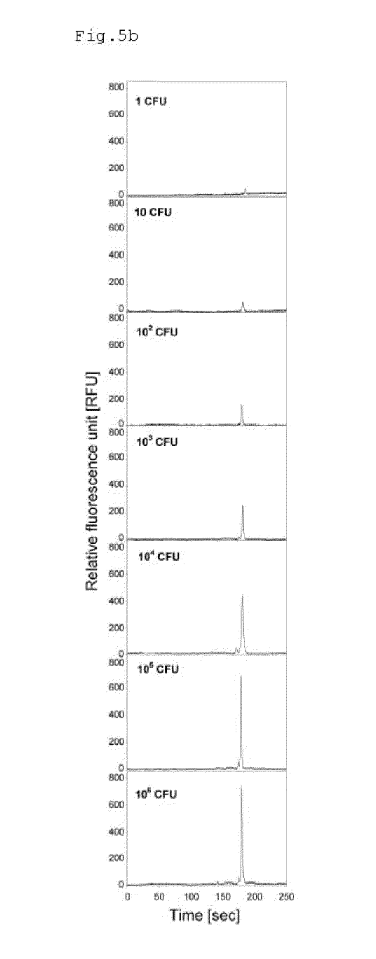

In this regard, FIG. 5 provides an electropherogram that shows a monoplex pathogen detection result. FIG. 5a shows a detection result of Staphylococcus aureus; FIG. 5b, a detection result of E. coli O157:H7; and FIG. 5c, a detection result of Salmonella typhimurium. FIG. 5 shows that as the concentration of target cell increase, higher peak intensity appears on the electropherogram. The elution times of the peaks were about 160 seconds, about 180 seconds, and about 200 seconds, respectively, which are matched with about 20-mer barcode DNA for Staphylococcus aureus, about 30-mer barcode DNA for E. coli O157:H7, and about 40-mer barcode DNA for Salmonella typhimurium. Those fluorescence peaks of the DNA barcode strands were produced in the electropherogram within about 5 minutes, which shows high speed of pathogen detection by the microdevice of the present disclosure.

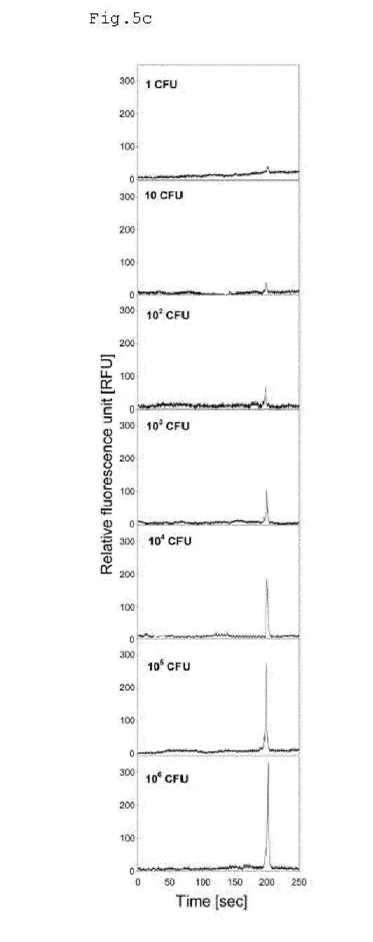

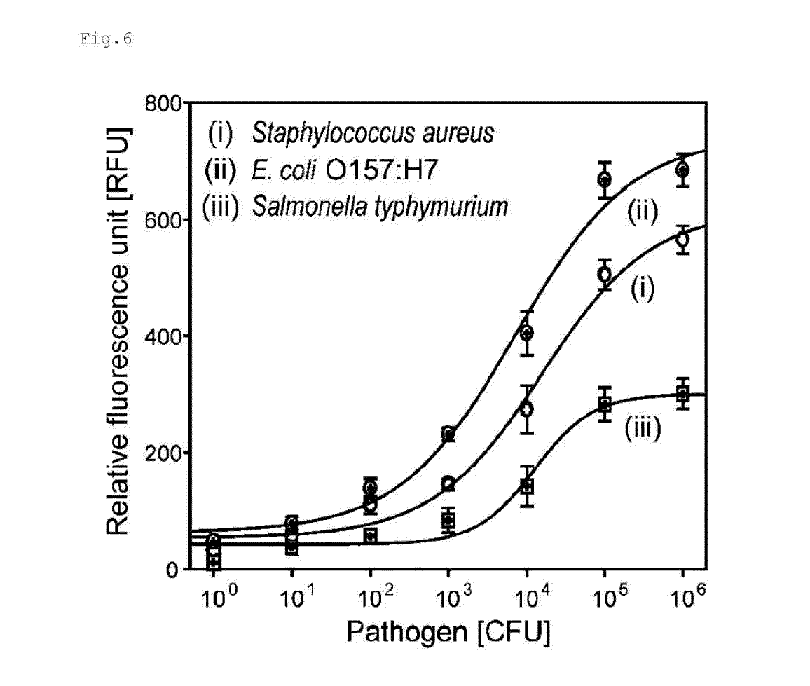

FIG. 6 is a graph showing RFU (Relative Fluorescent Unit) values corresponding to concentrations of the target pathogens in the monoplex pathogen detection in accordance with an illustrative embodiment. The graph reveals a sigmoidal relationship, and the dynamic range of each pathogen was set to be about 1 CFU to about 10⁶ CFU. Table 1 provides RFU values dependent on an input cell number in the monoplex pathogen detection, and Table 2 shows sigmodial equations for the quantitative analysis of pathogens.

TABLE 1

| | RFU of | RFU of | RFU of |
|---|---|---|---|
| | Staphylococcus | Escherichia coli | Salmonella |
| CFU | aureus | O157:H7 | typhimurium |
| 1 | 31 ± 4.8 | 46 ± 6.2 | 11 ± 4.8 |
| 10 | 55 ± 12 | 77 ± 13 | 37 ± 9.7 |
| 10² | 109 ± 15 | 138 ± 17 | 56 ± 11.4 |
| 10³ | 145 ± 9 | 231 ± 11 | 83 ± 21.5 |
| 10⁴ | 274 ± 41 | 404 ± 38 | 142 ± 34.2 |
| 10⁵ | 505 ± 26 | 667 ± 30 | 283 ± 28.9 |
| 10⁶ | 565 ± 24 | 684 ± 28 | 301 ± 25.7 |

TABLE 2

| Targets | Staphylococcus aureus | Escherichia coli O157:H7 | Salmonella typhimurium |
|---|---|---|---|
| Sigmoidal equation | $\begin{matrix}y = 53.07 + \frac{563.11}{1 + {e}^{- ( \frac{x - 15560.08}{- 0.62} )}} \\ ( {R}^{2} = 0.9899 )\end{matrix}$ | $\begin{matrix}y = 62.18 + \frac{680.46}{1 + {e}^{- ( \frac{x - 7155.018}{- 0.59} )}} \\ ( {R}^{2} = 0.9897 )\end{matrix}$ | $\begin{matrix}y = 41.55 + \frac{259.34}{1 + {e}^{- ( \frac{x - 12583.19}{- 1.1567} )}} \\ ( {R}^{2} = 0.9661 )\end{matrix}$ |

y: RFU and x: cell number

According to the data of Table 1, the fluorescence intensities at about 1 CFU were found to be about 31±4.8 RFU for Staphylococcus aureus, about 46±6.2 RFU for E. coli O157:H7, and about 11±4.8 RFU for Salmonella typhimurium. These values are clearly distinguishable from a background noise of about 1.92±0.65 RFU, which indicates that single cell detection was successfully performed.

Note that the total analysis time was less than about 30 minutes. To elaborate, it took about 20 minutes for immuno-reaction, less than about 5 minutes for magnetic separation and barcode DNA dehybridization, and less than about 5 minutes for CE separation and detection. From this result, it was proved that the microdevice of the present disclosure enables more rapid analysis as compared to the case of using conventional analysis methods.

### 7. Multiplex Pathogen Detection

To evaluate selectivity and multiplexing capability for pathogen detection on the microdevice of the present disclosure, four tests were conducted for different combinations of target pathogens where three sets of particle probes were all present.

To elaborate, the present inventors systematically combined two types of target pathogens (Staphylococcus aureus+E. coli O157:H7, Staphylococcus aureus+Salmonella typhimurium, and E. coli O157:H7+Salmonella typhimurium) as well as all the three target pathogens (Staphylococcus aureus+E. coli O157:H7+Salmonella typhimurium) under the same condition that an input cell number was set to about 10⁵ CFU.

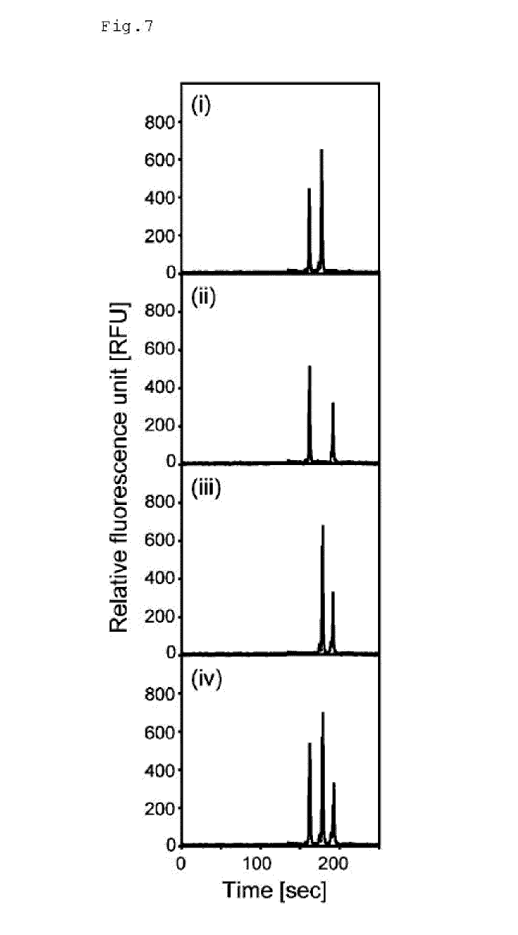

FIG. 7 is a graph showing measurements of RFU (Relative fluorescence unit) values with the lapse of time when the concentration of each pathogen is about 10⁵ CFU in an experiment for the multiplex pathogen detection. Specific multiplex pathogens are: (i) Staphylococcus aureus+E. coli O157:H7, (ii) Staphylococcus aureus+Salmonella typhimurium, (iii) E. coli O157:H7+Salmonella typhimurium, (iv) Staphylococcus aureus+E. coli O157:H7+Salmonella typhimurium.

As can be seen from FIG. 7, all the peaks were found to appear at elution times with high signal-to-noise ratios. This result indicates that the presence of target pathogens was accurately demonstrated. Here, importantly, only target specific barcode DNAs from particle-pathogen immuno-complexes were detected, although all the particle probes coexisted. This result implies that specific cross-immunobinding did not occur between the particle probes and the pathogens. Differences in fluorescence signal intensities of the respective target bacteria are deemed to be related to other binding constants between antibodies corresponding to the pathogens.

These results imply that more improved multiplexing analysis can be conducted by using the microdevice of the present disclosure by adjusting the lengths of DNA barcodes for the target pathogens and optimizing the design of the CE microchannel design.

### 8. LOD (Limit of Detection) Test

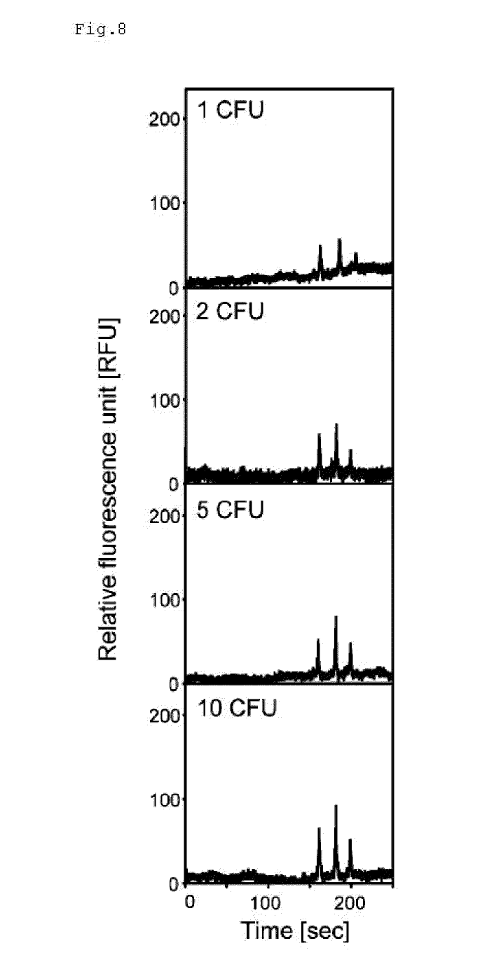

Detection limit of pathogen is an important issue in biosafety screening and early diagnosis in biomedical clinics. The capability of pathogen detection with small cell numbers may allow omission of tedious and time-consuming culturing steps. In this regard, the present inventors performed a LOD test for triplex pathogen detection in the microdevice by using the three target pathogens and all the particle probes. In this test, the input cell number was controlled to be about 1 CFU, about 2 CFU, about 5 CFU, and about 10 CFU, and the resultant electropherogram is shown in FIG. 8.

Referring to FIG. 8, even at an extremely low concentration of input cells, all the peaks corresponding to the respective target pathogens were successfully observed. Peaks on the graph from the left indicate the presence of Staphylococcus aureus, E. coli O157:H7, and Salmonella typhimurium in order. Here, note that multiple fluorescence peak signals at the single-cell level were clearly distinguishable from a background signal, which implies that the multiplex single cell pathogen detection can be performed by the microdevice in accordance with the present disclosure. An average signal-to-noise ratio was about 19.7±3.05 for Staphylococcus aureus, about 28.4±3.81 for E. coli O157:H7, and about 4.3±1.87 for Salmonella typhimurium, respectively. The large number of barcode DNA strands on each AuNP (i.e., about 1.11×10⁴ for Staphylococcus aureus, about 1.13×10⁴ for E. coli O157:H7, and about 1.31×10⁴ for Salmonella typhimurium) were successfully detectable on the microdevice in combination of a laser-induced fluorescence detection system. That is, the amount of the DNA barcode strands (˜10⁴) per AuNP is sufficient enough to be detected in the laser-induced confocal fluorescence detector, and it is possible to perform analysis at a single cell level.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

What is claimed is:

1. A microdevice for biomaterial detection, comprising:
a passive micromixer to mix a biomaterial, a first probe, and a second probe;
a magnetic separation chamber connected with the passive micromixer; and
a capillary electrophoresis channel connected with the magnetic separation chamber.

2. The microdevice for biomaterial detection of claim 1, wherein the biomaterial can be detected by using its specific antibody.

3. The microdevice for biomaterial detection of claim 1, wherein the first probe includes a magnetic microparticle probe.

4. The microdevice for biomaterial detection of claim 3, wherein the magnetic microparticle probe includes at least one specific antibody for the biomaterial, the specific antibody being immobilized at a surface of the magnetic microparticle probe.

5. The microdevice for biomaterial detection of claim 1, wherein the second probe includes a nanoparticle of gold, silver, platinum, palladium, copper, nickel, zinc, or silicon oxide.

6. The microdevice for biomaterial detection of claim 5, wherein the nanoparticle includes a specific antibody for the biomaterial and at least one barcode polymer, each of the specific antibody and the barcode polymer being immobilized at a surface of the nanoparticle.

7. The microdevice for biomaterial detection of claim 6, wherein the barcode polymer has a negative charge and is available to be separated according to its size by using a capillary electrophoresis.

8. The microdevice for biomaterial detection of claim 1, wherein the passive micromixer has an intestine-shaped structure including at least one corner and a tooth-shaped projection, and a centrifugal force generated at the corner can improve a mixing efficiency of the passive micromixer.

9. The microdevice for biomaterial detection of claim 1, wherein the passive micromixer mixes the biomaterial, the first probe, and the second probe to form a complex of first probe-biomaterial-second probe.

10. The microdevice for biomaterial detection of claim 9, wherein the magnetic separation chamber separates a part of the complex of first probe-biomaterial-second probe by applying a magnetic field.

11. The microdevice for biomaterial detection of claim 10, wherein the capillary electrophoresis channel quantitatively detects the part of the complex of first probe-biomaterial-second probe separated in the magnetic separation chamber by using a capillary electrophoresis.

12. The microdevice for biomaterial detection of claim 1, wherein the microdevice further includes a sample inlet at a upstream of the passive micromixer, and the biomaterial, the first probe, and the second probe are introduced into the microdevice through the sample inlet.

13. The microdevice for biomaterial detection of claim 1, wherein the microdevice further includes a sample reservoir and a waste reservoir which are respectively connected with the magnetic separation chamber, and a cathode reservoir and an anode reservoir which are respectively connected with the capillary electrophoresis channel.

14. The microdevice for biomaterial detection of claim 1, wherein the microdevice can be used for a monoplex biomaterial detection for one kind of biomaterial by using a single-sized barcode polymer, or a multiplex biomaterial detection for at least two kinds of biomaterials by using differently-sized barcode polymers.

15. The microdevice for biomaterial detection of claim 1, wherein a total analysis time required from sample pretreatment to biomaterial detection by using the microdevice is about 30 minutes or less.

16. The microdevice for biomaterial detection of claim 1, wherein the microdevice performs the detection at a single-cell level.
